(19) United States
(12) Patent Application Publication
(10) Pub. No.: US 2007/0231363 A1
(43) Pub. Date: Oct. 4, 2007

(54) Coatings formed from stimulus-sensitive material

(76) Inventors: Yung-Ming Chen, Cupertino, CA (US); Yiwen Tang, San Jose, CA (US); Stephen D. Pacetti, San Jose, CA (US); Lothar Kleiner, Los Altos, CA (US)

Correspondence Address: SQUIRE, SANDERS & DEMPSEY LLP, 1 MARITIME PLAZA, SUITE 300, SAN FRANCISCO, CA, 94111, US

(21) Appl. No.: 11/394,295

(22) Filed: Mar. 29, 2006

(51) Int. Cl. A61K 38/48; A61K 35/12; A61K 31/56; A61K 31/573; A61K 31/551; A61K 31/4745; A61K 31/445; A61K 31/337; A61F 2/02

(52) U.S. Cl. 424/423; 424/93.7; 514/171; 514/291; 514/327; 514/449; 514/211.08; 424/94.64

(57) ABSTRACT

A coating comprising a stimulus-responsive material and a bioactive agent for controlled release of the bioactive agent and methods of making and using the same are disclosed.

### FIELD OF THE INVENTION

The present invention generally relates to coatings on an implantable medical device for treating adverse side effects related to implantation of the medical device.

### BACKGROUND OF THE INVENTION

Stents are used not only as a mechanical intervention in vascular conditions, but also as a vehicle for providing biological therapy. As a mechanical intervention, stents act as scaffoldings, functioning to physically hold open and, if desired, to expand the wall of the passageway. Typically, stents are capable of being compressed, so that they can be inserted through small vessels via catheters, and then expanded to a larger diameter once they are at the desired location. Examples in patent literature disclosing stents that have been applied in PTCA (Percutaneous Transluminal Coronary Angioplasty) procedures include stents illustrated in U.S. Pat. No. 4,733,665 issued to Palmaz, U.S. Pat. No. 4,800,882 issued to Gianturco, and U.S. Pat. No. 4,886,062 issued to Wiktor.

Biological therapy can be achieved by medicating the stents. Medicated stents locally administer a therapeutic substance at the diseased site. In order to provide an effective concentration at the treated site, systemic administration of such medication often produces adverse or toxic side effects on the patient. Local delivery is a preferred method of treatment in that smaller total levels of medication are administered in comparison to systemic dosages, but are concentrated at a specific site. Local delivery thus-produces fewer side effects and achieves better results.

However, stenting may result in undesirable side effects. Such undesirable side effects include, for example, restenosis, thrombosis, etc. For example, angioplasty induces localized injury to the vessel wall, which leads to the release of vasoactive, thrombogenic, and mitogenic factors that result in processes causing re-narrowing (restenosis) at the injured site. Thrombosis is the formation of a blood clot at the treatment site. Placement of a metal stent in a vessel gives rise to a blood-metal interface; this interface causes platelet deposition, which is responsible for the significant thrombotic potential of coronary stents.

Therefore, there is a need for medical devices that produce reduced or minimal undesirable side effects upon implantation.

The embodiments described below address the above needs and issues.

### SUMMARY OF THE INVENTION

The present invention provides a coating on a medical device that includes a stimulus-sensitive material. The coating can include a bioactive agent such as a cell or a drug. Upon exposure to a stimulus (for example, a heat or pH change), the stimulus-sensitive material can undergo a property change that changes the release rate of a bioactive agent (e.g., a drug) from a coating. A property change that falls within the scope of the present invention can be, for example, a change of reversible bulk properties or reversible swelling behavior (e.g., hydrogels). Some examples of the bioactive agent include, but are not limited to, paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), pimecrolimus, imatinib mesylate, midostaurin, clobetasol, bioactive RGD, CD-34 antibody, abciximab (REOPRO), progenitor cell capturing antibody, prohealing drugs, prodrugs thereof, co-drugs thereof, or a combination thereof.

A medical device having a coating described herein can be used to treat, prevent, or ameliorate a vascular medical condition. Some exemplary vascular medical conditions include atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

### BRIEF DESCRIPTION OF THE DRAWINGS

FIG. 1 shows a stent that can be used in accordance with some embodiments of the present invention;

FIG. 2 shows a stent strut with micro-channels on an outer surface of the stent strut, which can be used in accordance with some embodiments of the present invention.

### DETAILED DESCRIPTION OF THE INVENTION

The present invention provides a coating on a medical device that includes a stimulus-sensitive material. The coating can include a bioactive agent such as a cell, peptide, protein, DNA, RNA, or a drug. Upon exposure to a stimulus (for example, a temperature or pH change), the stimulus-sensitive material can undergo a property change that changes the release rate of a bioactive agent (e.g., a drug) from a coating. A property change that falls within the scope of the present invention can be, for example, a change of reversible bulk properties or reversible swelling behavior (e.g., hydrogels). Some examples of the bioactive agent include, but are not limited to, paclitaxel, docetaxel, estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N-1-tetrazolyl)-rapamycin (ABT-578), pimecrolimus, imatinib mesylate, midostaurin, clobetasol, bioactive RGD, CD-34 antibody, abciximab (REOPRO), progenitor cell capturing antibody, prohealing drugs, prodrugs thereof, co-drugs thereof, or a combination thereof.

A medical device having a coating described herein can be used to treat, prevent, or ameliorate a vascular medical condition. Some exemplary vascular medical conditions include atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, and combinations thereof.

### Stimulus-Sensitive Material

The implantable medical device of the present invention can incorporate at least one stimulus-sensitive material and at least one bioactive agent. As used herein, the term “stimulus-sensitive material” is used interchangeably with the term “stimulus-responsive material”. The term “bioactive agent” is sometimes referred to as “therapeutic agent.”

A stimulus-sensitive material is a material that senses and responds to a physical or chemical stimulus in its local environment in a controlled and reproducible manner. A physical stimulus can be, but is not limited to, heat (e.g., externally applied heat or heat from a local temperature increase at a site of implantation of a device), electrical field, pressure, sound or radiation. A chemical stimulus can be, but is not limited to, a change in the pH, ionic strength or oxidative environments in the local environment of the stimulus-responsive material. An example of a stimulus-responsive material is a synthetic material such as a polymer. In some embodiments, the polymer can be a biostable polymer (e.g., an acrylate and/or methacrylate polymer) or a bioabsorbable polymer.

In some embodiments, the stimulus-responsive material is a thermo-responsive polymer. Thermo-responsive polymers, along with pH sensitive polymers, are sometimes referred to as “smart polymers”. See, for example, U.S. Pat. Nos. 4,830,855 to Stewart, 5,120,349 to Stewart et al., 5,665,822 to Bitler et al., 6,199,318 to Stewart et al., 6,540,984 to Stewart et al., 6,492,462 to Bitler et al., 6,548,132 to Clarke et al.

In some embodiments, a coating including a smart polymer can control the delivery of endothelial cells for preventing or reducing thrombosis of coronary stenting. Thrombosis has increased as the use of stents for broad spectrums of lesions has increased. Endothelial cells on a coating surface can reduce the incidence of thrombosis (see. e.g., Marcus, et al., Atherioscler. Thromb. Vasc. Biol. 21:178-182 (2001); Scott, et al., Am. Heart J. 129(5):860-866 (1995)). A medical device can be made with a smart polymer coating as the topcoat on a layer of endothelial cells. The topcoat can protect the endothelial cells from delaminating from the medical device surface during deployment. Upon implantation, a body's temperature can trigger a change in the smart polymer's physical properties (e.g., hydrophilicity, permeability, etc) to allow the endothelial cells to be released thus providing local delivery of the cells.

In some embodiments, a medical device having a coating that includes a smart polymer described herein can be used to regulate local delivery of an agent (e.g., a drug) to the implantation site of the medical device. For example, coronary stenting can also be a strong inflammatory stimulus. Post-stenting inflammation can cause the arterial wall temperature to increase (see, e.g., Diamantopoulos, et al., J. Invasive Cardiol. 15(4):191-7 (2003)). A local temperature increase (e.g., a raise of 0.5° C.) at the wall surfaces (hotspots) and a higher local temperature in the tissue near the inflammation site has been reported in some human trials; this was attributed to greater regional macrophage activity. (see, e.g., Stefanandis C., et al., J. Am. Coll. Cardiol. 37(5):1277-1283 (2001)). The temperature increase of an injured site allows the design of a coating that includes a smart polymer to serve as a switch to regulate (switch on/off) the release of an agent (e.g., a drug) from a coating such that the coating will not release the agent or drug at the normal body temperature (about 37° C.), but will release the agent or drug or have different release profiles at injured sites because the temperature at those sites is greater than normal body temperature (e.g., greater than 37° C.). Tuning of the phase transition temperature of the stimulus-sensitive polymer can be achieved by incorporating different molecular moieties as the polymeric side chains or, in some embodiments, by blending different grades of a stimulus-sensitive polymer, one example of which is Intelimer (available from Landec Corporation, Menlo Park, Calif.).

Incorporating a crystallizable side chain onto the polymer backbone can impact the properties of a thermo-responsive polymer. Thus, in response to a thermal stimulus, the polymer can undergo a reversible physical change, which is accompanied by a reversible change in bulk properties or swelling behavior attributable to its crystallizable side chains. Thermo-responsive polymers can abruptly change properties such as permeability, adhesion, or viscosity when subjected to small temperature fluctuations at the medical device's implantation site (e.g., vessel wall). In some embodiments, these temperature fluctuations result from a biological response (for example, inflammatory response, or immune response). In some embodiments, the stimulus-responsive polymers change permeability when exposed to a physical or chemical stimulus, i.e. the polymer undergoes a phase change, for example, from a homogeneous phase to a hydrogel having pores. The overall result in these embodiments is that the stimulus can cause the polymer to reversibly form pores through which the drug in the coating can diffuse. Thus regulating the drug flux at the corresponding tissue based on the tissue's biological needs, and thus, the polymer can provide a tuned therapeutic effect during the healing process. For example, to put it more concretely, local inflammation at the implantation site leads to a temperature increase. This temperature increase causes the stimulus-responsive polymer to change phase to a more permeable or porous form. For example, the phase change from semi-crystalline to amorphous can result in the increased flexibility of the polymer chain by several orders of magnitude and hence permeability also changes by several orders of magnitude (see, e.g., Z. Mogri and D. R. Paul, Polymer, 42, 2531 (2001); Hedenqvist, M. and Gedde, U. W., Prog. Polym. Sci., 21:299-333 (1996) (Review); Z. Mogri and D. R. Paul, J. Membrane Sci., 175, 253 (2000)). If the drug is an anti-inflammatory agent, it alleviates the local inflammation. This can lead to a decrease in temperature accompanied by a change in polymer phase to a less permeable form that decreases the antiinflammatory dosage at the implantation site. Those of ordinary skill in the art would recognize that this describes just one example of a myriad of stimulus-induced phase changes that can occur in polymer systems.

In some embodiments, the temperature fluctuation can be in a range of, for example, from about 36.5° C. to 38.5° C. The changes triggered by a temperature fluctuation can be within a range compatible with many biological applications. While advantageous with respect to their reversible properties, these thermo-responsive polymers can have an orderly structure at temperatures below their side-chain melting temperatures, which can sometimes cause the polymers to be brittle although, in some embodiments, these thermo-responsive polymers can have non-brittle behavior at temperatures below their side-chain melting temperatures.

In some embodiments, an external temperature control system can cause or regulate the temperature change. Such a temperature control system can use a physical stimulus, such as ultrasonic energy, or magnetic or electrical fields. For example, the temperature of an implanted metallic stent with the thermo-responsive coating can be altered by external application of an oscillating electrical field to induce eddy currents, and thus heat the metal.

In some embodiments, the polymer's thermal properties, or response to a temperature change, can be modified or tuned by adjusting the side chain length or by changing different functional groups. Exemplary side chain lengths can be from C12 to C24. The melting point of some alkane moieties is shown in Table 1.

TABLE 1
Melting points of some alkanes

| Alkane | Chemical Formula | Melting points (° C.) |
|---|---|---|
| Eicosane | C₂₀H₄₂ | 36.8 |
| Heneicosane | C₂₁H₄₄ | 40.5 |
| Docosane | C₂₂H₄₆ | 44.4 |
| Tricosane | C₂₃H₄₈ | 47.6 |
| Tetracosane | C₂₄H₅₀ | 50.9 |
| Octadecyl cyclohexane | C₂₄H₄₈ | 41.6 |

Exemplary functional groups can be amino, ether, ester, hydroxyl, sulfhydryl, amide, alkene, alkyne, phenyl, carboxyl, sulfate, phosphate and phosphonate.

### Coating Constructs

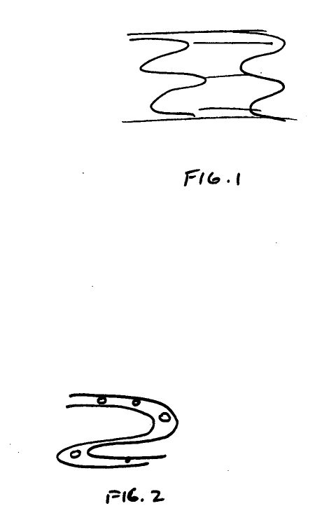

In some embodiments, a combination of a stimulus-responsive material(s) and a bioactive agent(s) described above can be used to make a drug delivery system of the present invention. For example, in some embodiments, a first layer of a bioactive agent (which in some embodiments can be a biological agent) and a second layer of a stimulus-responsive material can be applied to a surface of a medical device (e.g., stent) (FIG. 1). In some embodiments, a composition including the bioactive agent and the stimulus-responsive material can be deposited within micro-depots or micro-channels on the surface of a medical device (e.g., stent). In other embodiments, the bioactive agent can mix with the stimulus-responsive material forming a suspension, which suspension is then dispersed into a polymer matrix for coating a medical device (e.g., stent). It should be understood that these methods can be used individually or combined to make the drug delivery system(s) of the present invention.

In some embodiments, the stimulus-responsive material can be blended with another polymer or polymers such as polyethylene adipate, SOLEF™ (poly-vinylidene fluoride and its copolymers), poly-ethylene glycol or poly-lactic acid or a combination thereof to expand the variety of applications to which the drug delivery system may be applied. Such applications include, for example, (a) preserving the biological cells or therapeutic agent(s), (b) modulating the absorption rate of degradable polymers, (c) modifying a material's surface by changing the hydrophobicity-hydrophilicity balance to regulate cell attachment or improve the material's biocompatibility or (d) micro-patterning the material to immobilize biological signaling molecules to regulate cell function.

In one embodiment, an implantable medical device, such as a stent, can be seeded with a layer of endothelial cells by methods known by those skilled in the art. A topcoat layer of a stimulus-responsive material can then be applied as a topcoat layer. The topcoat layer can serve at least two purposes, (1) reduction or elimination of endothelial cell layer delamination once the medical device is implanted in a target vessel, and (2) sustained release of the endothelial cells to the target vessel. In some embodiments, the stimulus-responsive material is thermo-responsive. Thus, when the device is delivered to a target vessel, a temperature increase in the target vessel due to inflammation caused by implantation of medical device in the target tissue (or vessel) can cause the polymer to undergo reversible change, which stimulates a change in physical properties of the thermo-responsive polymer to allow the endothelial cells to be released in a sustained manner. In this embodiment, the stimulus-source, i.e., the body's own temperature, is internal. In some embodiments, the stimulus-source can be external to the target vessel, such as application of a focused oscillating electric field, magnetic field, or ultrasonic field. In some embodiments, the polymer can be an acrylate or methacrylate with short, crystallizable side chains. In such embodiments, the glass transition temperature (“T_{g}”) and the melting temperature (“Tₘ”) of the side chain crystalline Tₘ and/or T_{g} of the polymer can have a range that can be narrow, e.g., in a range between about 1° C. and 10° C., typically about 3° C.

In yet another embodiment, an implantable medical device, such as a stent, can be coated with a composition including a stimulus-responsive material, a bioactive agent and/or a biocompatible polymer, or any combination thereof. In some embodiments, the stimulus-responsive material can be a thermo-responsive polymer, such as acrylate, methacrylate or a derivative thereof. Additionally, the side chain(s) of the thermo-responsive polymer can be heat manipulated to affect the reversible properties of the polymer. In some embodiments, the drug can be everolimus, clobetasol or a combination thereof. As discussed previously, coronary stenting has been shown to cause an increase in the arterial wall temperature of the target vessel. Thus, in this embodiment, the bioactive agent can be released in areas in which the arterial wall temperature is higher than comparable, normal, non-stented vessel. The drug delivery system can be designed to have a normal drug release profile or no release profile at normal body temperatures (37° C.), while having a different release profile for injured areas of the target vessel by blending different thermo-responsive polymers or by incorporating different side chains to the polymer itself.

In yet another embodiment, an implantable medical device, such as a stent with micro-channels, can be coated with a composition including a stimulus-responsive material, a bioactive agent and/or a biocompatible polymer, or any combination thereof. In this embodiment, the composition can be placed in micro-channels, which lie in low strain regions of the stent (see FIG. 2). As a result, the brittle behavior exhibited by the smart polymer below the side-chain melting temperature is not a limitation as the polymer is not subjected to high strains.

In yet another embodiment, an implantable medical device, such as a stent, can be coated with microspheres having a composition including a stimulus-responsive material, a bioactive agent and/or a biocompatible polymer, or any combination thereof. In some embodiments, the bioactive agent can be encapsulated in the polymer microspheres. These microspheres can in turn be dispersed in another polymer matrix, such as a bioabsorbable polymer, biopolymer or biostable polymer. As a result, the brittle behavior exhibited by the smart polymer below the side-chain melting temperature can be controlled in the coating process. Alternatively, the brittle behavior of the smart polymer can be controlled by controlling the molecular weight of the back-bone in that a higher molecular weight of smart polymer can cause the polymer to become less brittle.

### Biocompatible Polymers

Any biocompatible polymer or polymeric material can be used along with the stimulus responsive material to form a coating on a medical device. The biocompatible polymer can be biodegradable (either bioerodable or bioabsorbable or both) or nondegradable, and can be hydrophilic or hydrophobic.

The polymer should be biocompatible, for example a polymeric material which, in the amounts employed, is non-toxic and chemically inert as well as substantially non-immunogenic and non-inflammatory, which for purposes of this document means that any immunogenic or inflammatory effect is not large enough to cause one of ordinary skill in the art to disqualify the polymer for use in an implantable medical device. A bioabsorbable polymer breaks down in the body and is not present sufficiently long after delivery to cause an adverse local response. Bioabsorbable polymers are gradually absorbed or eliminated by the body by hydrolysis, bulk, or surface erosion, and metabolic processes. A biostable polymer does not break down in the body, and thus a biostable polymer is present in the body for a substantial amount of time after delivery.

Representative biocompatible polymers include, but are not limited to, poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) PEO/PLA)), polyalkylene oxides such as poly(ethylene oxide), poly(propylene oxide), poly(ether ester), polyalkylene oxalates, phosphoryl choline, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, PARYLENE, PARYLENE-C, PARYLAST, polyethylene, polyethlyene terephthalate, or combinations thereof. In some embodiments, the coating described herein can exclude any one of the aforementioned polymers.

As used herein, the terms poly(D,L-lactide), poly(L-lactide), poly(D,L-lactide-co-glycolide), and poly(L-lactide-co-glycolide) can be used interchangeably with the terms poly(D,L-lactic acid), poly(L-lactic acid), poly(D,L-lactic acid-co-glycolic acid), or poly(L-lactic acid-co-glycolic acid), respectively.

### Biobeneficial Material

In some embodiments, the biocompatible polymer or polymeric material described above can include a biobeneficial material. The biobeneficial material can be a polymeric material or non-polymeric material. The biobeneficial material is preferably non-toxic, non-antigenic and non-immunogenic. A biobeneficial material is one which enhances the biocompatibility of the particles or device by being non-fouling, hemocompatible, actively non-thrombogenic, or antiinflammatory, all without depending on the release of a pharmaceutically active agent.

Representative biobeneficial materials include, but are not limited to, polyethers such as poly(ethylene glycol), copoly(ether-esters) (e.g. PEO/PLA), polyalkylene oxides such as poly(ethylene oxide), poly(propylene oxide), poly(ether ester), polyalkylene oxalates, polyphosphazenes, phosphoryl choline, choline, poly(aspirin), polymers and co-polymers of hydroxyl bearing monomers such as hydroxyethyl methacrylate (HEMA), hydroxypropyl methacrylate (HPMA), hydroxypropylmethacrylamide, poly(ethylene glycol)acrylate (PEGA), PEG methacrylate, 2-methacryloyloxyethylphosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as fibrin, fibrinogen, cellulose, starch, collagen, dextran, dextrin, hyaluronic acid, fragments and derivatives of hyaluronic acid, heparin, fragments and derivatives of heparin, glycosamino glycan (GAG), GAG derivatives, polysaccharide, elastin, chitosan, alginate, silicones, PolyActive™, and combinations thereof. In some embodiments, a coating described herein can exclude any one of the aforementioned polymers.

The term PolyActive™ refers to a block copolymer having flexible poly(ethylene glycol) and poly(butylene terephthalate) blocks (PEGT/PBT). PolyActive™ is intended to include AB, ABA, BAB copolymers having such segments of PEG and PBT (e.g., poly(ethylene glycol)-block-poly(butyleneterephthalate)-block poly(ethylene glycol) (PEG-PBT-PEG).

In a preferred embodiment, the biobeneficial material can be a polyether such as poly(ethylene glycol) (PEG) or polyalkylene oxide.

### Bioactive Agents

A coating including a stimulus responsive material can include any bioactive agent, which can be a therapeutic, prophylactic, or diagnostic agent. These agents can have antiproliferative or antiinflammatory properties or can have other properties such as antineoplastic, antiplatelet, anticoagulant, antifibrin, antithrombotic, antimigratory, antimitotic, antibiotic, antiallergic, and antioxidant. The agents can be cystostatic agents, agents that promote the healing of the endothelium such as NO releasing or generating agents, agents that attract endothelial progenitor cells, or agents that promote the attachment, migration and proliferation of endothelial cells (e.g., natriuretic peptide such as CNP, ANP or BNP peptide or an RGD or cRGD peptide), while quenching smooth muscle cell proliferation. Examples of suitable therapeutic and prophylactic agents include synthetic inorganic and organic compounds, proteins and peptides, polysaccharides and other sugars, lipids, and DNA and RNA nucleic acid sequences having therapeutic, prophylactic or diagnostic activities. Some other examples of the bioactive agent include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy. Examples of antiproliferative agents include rapamycin and its functional or structural derivatives, 40-O-(2-hydroxy)ethyl-rapamycin (everolimus), and its functional or structural derivatives, paclitaxel and its functional and structural derivatives. Examples of rapamycin derivatives include 40-epi-(N1-tetrazolyl)-rapamycin (ABT-578), 40-O-(3-hydroxy)propyl-rapamycin, 40-O-[2-(2-hydroxy)ethoxy]ethyl-rapamycin, and 40-O-tetrazole-rapamycin. Examples of paclitaxel derivatives include docetaxel. Examples of antineoplastics and/or antimitotics include methotrexate, azathioprine, vincristine, vinblastine, fluorouracil, doxorubicin hydrochloride (e.g. Adriamycin® from Pharmacia & Upjohn, Peapack N.J.), and mitomycin (e.g. Mutamycin® from Bristol-Myers Squibb Co., Stamford, Conn.). Examples of such antiplatelets, anticoagulants, antifibrin, and antithrombins include sodium heparin, low molecular weight heparins, heparinoids, hirudin, argatroban, forskolin, vapiprost, prostacyclin and prostacyclin analogues, dextran, D-phe-pro-arg-chloromethylketone (synthetic antithrombin), dipyridamole, glycoprotein IIb/IIIa platelet membrane receptor antagonist antibody, recombinant hirudin, thrombin inhibitors such as Angiomax (Biogen, Inc., Cambridge, Mass.), calcium channel blockers (such as nifedipine), colchicine, fibroblast growth factor (FGF) antagonists, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of antiinflammatory agents including steroidal and non-steroidal antiinflammatory agents include tacrolimus, dexamethasone, clobetasol, or combinations thereof. Examples of cytostatic substances include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents which may be appropriate include alpha-interferon, pimecrolimus, imatinib mesylate, midostaurin, γ-hiridun (which can be a thrombin inhibitor), bioactive RGD, and genetically engineered endothelial cells. The foregoing substances can also be used in the form of prodrugs or co-drugs thereof. The foregoing substances also include metabolites thereof and/or prodrugs of the metabolites. The foregoing substances are listed by way of example and are not meant to be limiting. Other active agents which are currently available or that may be developed in the future are equally applicable. In some embodiments, a coating described herein can exclude any of the above agents.

The dosage or concentration of the bioactive agent required to produce a favorable therapeutic effect should be less than the level at which the bioactive agent produces toxic effects and greater than the level at which non-therapeutic results are obtained. The dosage or concentration of the bioactive agent can depend upon factors such as the particular circumstances of the patient, the nature of the trauma, the nature of the therapy desired, the time over which the ingredient administered resides at the vascular site, and if other active agents are employed, the nature and type of the substance or combination of substances. Therapeutically effective dosages can be determined empirically, for example by infusing vessels from suitable animal model systems and using immunohistochemical, fluorescent or electron microscopy methods to detect the agent and its effects, or by conducting suitable in vitro studies. Standard pharmacological test procedures to determine dosages are understood by one of ordinary skill in the art.

### Examples of Implantable Device

As used herein, an implantable device can be any suitable medical substrate that can be implanted in a human or veterinary patient. Examples of such implantable devices include self-expandable stents, balloon-expandable stents, stent-grafts, grafts (e.g., aortic grafts), heart valve prostheses, cerebrospinal fluid shunts, pacemaker electrodes, catheters, and endocardial leads (e.g., FINELINE and ENDOTAK, available from Guidant Corporation, Santa Clara, Calif.), anastomotic devices and connectors, orthopedic implants such as screws, spinal implants, electro-stimulatory devices. The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium, or combinations thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. Devices made from bioabsorbable or biostable polymers could also be used with the embodiments of the present invention.

### Method of Use

In accordance with embodiments of the invention, a coating subjected to the treatment of a phase inversion process described above can be used to provided controlled release of a bioactive agent from a medical device (e.g., stent) during delivery and (in the case of a stent) expansion of the device, or thereafter, at a desired rate and for a predetermined duration of time at the site of implantation.

Preferably, the medical device is a stent. The stent described herein is useful for a variety of medical procedures, including, by way of example, treatment of obstructions caused by tumors in bile ducts, esophagus, trachea/bronchi and other biological passageways. A stent having the above-described coating is particularly useful for treating diseased regions of blood vessels caused by artherosclerosis, lipid deposition, monocyte or macrophage infiltration, or dysfunctional endothelium or a combination thereof, or occluded regions of blood vessels caused by abnormal or inappropriate migration and proliferation of smooth muscle cells, thrombosis, and restenosis. Stents with coatings that are thermo-responsive can be placed in a wide array of blood vessels, both arteries and veins. Representative examples of sites include the iliac, renal, carotid and coronary arteries.

For implantation of a stent, an angiogram is first performed to determine the appropriate positioning for stent therapy. An angiogram is typically accomplished by injecting a radiopaque contrast agent through a catheter inserted into an artery or vein as an x-ray is taken. A guidewire is then advanced through the lesion or proposed site of treatment. Over the guidewire is passed a delivery catheter which allows a stent in its collapsed configuration to be inserted into the passageway. The delivery catheter is inserted either percutaneously or by surgery into the femoral artery, brachial artery, femoral vein, or brachial vein, and advanced into the appropriate blood vessel by steering the catheter through the vascular system under fluoroscopic guidance. A stent having the above-described features may then be expanded at the desired area of treatment. A post-insertion angiogram may also be utilized to confirm appropriate positioning.

### EXAMPLES

### Example 1

25 μg of everolimus, 15 μg of clobetasol and 120 μg of poly(octadecylmethacrylate) polymer are blended using conventional blending methods. The composition is then placed within microchannels of a 12 mm stent. The melting temperature of the polymer is 40° C. The release rate of the everolimus and clobetasol will therefore be regulated by the local environment of the vessel. Alternatively, the release rate of the everolimus and clobetasol can be regulated by an external source such as ultrasonic energy or induction heating by external application of an oscillating electromagnetic field.

### Example 2

Clobetasol is combined in a poly(hexadecyl acrylate) polymer in a 1:3 ratio by weight. The mixture of clobetasol and polymer is dissolved in methylene chloride. This solution is added to an aqueous solution containing 0.5% Pluronic F68 forming an oil-in-water emulsion. After sonication and evaporation of the solvent, microspheres of 0.5-50μ are obtained. These microspheres are dispersed in a solution of platinum-cured siloxane dissolved in heptane at a weigh ratio of 1:4 microspheres:silicone. The dispersion is applied via a direct application method onto the abluminal surface of a 12 mm stent so that the total clobetasol amount is 25 μg.

While particular embodiments of the present invention have been shown and described, it will be obvious to those skilled in the art that changes and modifications can be made without departing from this invention in its broader aspects. Therefore, the appended claims are to encompass within their scope all such changes and modifications as fall within the true spirit and scope of this invention.

What is claimed is:

1. An implantable medical device comprising a coating that comprises a stimulus-responsive material and at least one bioactive agent.

2. The medical device of claim 1 wherein the stimulus-responsive material is a material that upon exposure to a stimulus undergoes a change of at least one physical or chemical property such that the release rate of the bioactive agent changes.

3. The medical device of claim 1 wherein the bioactive agent is an antiproliferative, antiinflammatory, immune-modulating, antimigratory, antineoplastic, antimitotic, antiplatelet, anticoagulant, antifibrin, antibiotic, antioxidant, antiallergic, or antithrombotic, or a pro-healing agent, or combinations of these.

4. The medical device of claim 1 wherein the bioactive agent is paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, or midostaurin, or prodrugs, co-drugs, or combinations of these.

5. The device of claim 1 wherein the bioactive agent comprises a layer of endothelial cells.

6. The device of claim 1 wherein the stimulus-responsive material comprises a polymer that is adapted to undergo a reversible physical transformation when exposed to a physical or chemical stimulus.

7. The device of claim 1 wherein the stimulus-responsive material comprises a biostable polymer, a bioabsorbable polymer, or a combination of these.

8. The device of claim 7 wherein the biostable polymer is an acrylate, a methacrylate, or a combination thereof.

9. The device of claim 6 wherein the stimulus-responsive material comprises a polyacrylate or polymethacrylate with crystallizable sidechains.

10. The device of claim 6 wherein the physical or chemical stimulus is heat, light, a pH change, an ionic strength change, oscillating electric field, magnetic field, electromagnetic field, pressure, ultrasound, radiation, or a combination of these.

11. The device of claim 10 wherein the stimulus is heat from a local temperature increase at a site of implantation of the device.

12. The device of claim 11 wherein the stimulus is external or internal or both.

13. The device of claim 6 wherein the coating comprises microspheres formed from the stimulus-responsive polymer encapsulating the bioactive agent.

14. The device of claim 1 wherein the medical device includes an outer surface comprising micro-channels or pores.

15. The device of claim 14 wherein the micro-channels or pores contain both the bioactive agent and the stimulus-responsive material.

16. A method of forming a coating on an implantable medical device comprising:
encapsulating at least one bioactive agent with at least one stimulus-responsive material to form microsphere(s);
dispersing the microspheres into a polymer matrix; and
applying the polymer matrix with the microspheres to form a coating on an implantable medical device.

17. The method of claim 16 wherein the bioactive agent is an antiproliferative, antiinflammatory or immune modulating, antimigratory, antineoplastic, antimitotic, antiplatelet, anticoagulant, antifibrin, antibiotic, antioxidant, antiallergic substances, or antithrombotic, or a pro-healing agent, or combinations of these.

18. The method of claim 16 wherein the bioactive agent is paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N-1-tetrazolyl)-rapamycin (ABT-578), γ-hiridun, clobetasol, pimecrolimus, imatinib mesylate, or midostaurin, or prodrugs, co-drugs, or combinations of these.

19. The method of claim 16 wherein the microspheres are capable of selectively releasing the bioactive agent in response to a physical or chemical stimulus.

20. The method of claim 16 wherein the physical stimulus is a thermal stimulus.

21. The method of claim 16 wherein the thermal stimulus is an external stimulus, an internal stimulus, or both.

22. A method of forming a coating on an implantable medical device comprising:
forming a layer of endothelial cells on the medical device, and
forming a layer of a stimulus-responsive material on top of the layer of the endothelial cells.

23. The method of claim 22 wherein the stimulus-responsive material comprises a thermo-responsive polymer.

24. The method of claim 23 wherein the thermo-responsive polymer comprises units derived from an acrylate, methacrylate or combinations of these.

25. A method of forming a coating on a medical device comprising forming a topcoat which comprises a first bioactive agent and a stimulus-responsive material.

26. The method of claim 25 wherein the coating further comprises a layer underneath the topcoat wherein the layer comprises a biocompatible polymer.

27. The method of claim 25 wherein the topcoat further comprises a biocompatible polymer, which is not the stimulus-responsive material.

28. The method of claim 26 wherein the layer comprises a second bioactive agent,
wherein the second bioactive agent can be the same as or different from the first bioactive agent.

29. The method of claim 25 wherein the first bioactive agent is an antiproliferative, antiinflammatory or immune modulating, antimigratory, antineoplastic, antimitotic, antiplatelet, anticoagulant, antifibrin, antibiotic, antioxidant, antiallergic substances, or antithrombotic, or a pro-healing agent, or a combination of these.

30. The method of claim 25 wherein the first bioactive agent is paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, or midostaurin, or prodrugs, co-drugs, or combinations of these.

31. The method of claim 26 wherein the first bioactive agent and the second bioactive agent are independently an antiproliferative, antiinflammatory or immune modulating, antimigratory, antineoplastic, antimitotic, antiplatelet, anticoagulant, antifibrin, antibiotic, antioxidant, antiallergic substances, or antithrombotic, or a pro-healing agent, of a combination of these.

32. The method of claim 26 wherein the first bioactive agent and the second bioactive agent are independently paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, or midostaurin, or prodrugs, co-drugs, or combinations of these.

33. The method of claim 25 wherein the stimulus-responsive material is responsive to heat, light, a pH change, an ionic strength change, oscillating electric field, magnetic field, electromagnetic field, pressure, ultrasound, radiation, or a combination of these.

34. A method of forming a coating on an implantable medical device comprising:
forming a first layer of coating on a first region of a medical device wherein the first layer comprises a biocompatible polymer and optionally a first bioactive agent, and
forming a second layer of coating on a second region of the medical device where in the second layer comprises a stimulus-responsive material and a second bioactive agent,
wherein the first region and the second region are separate from each other or overlap,
wherein, if the first region overlaps with the second region, at least part of the second layer of coating is formed on top of a part of the first layer of coating,
wherein stimulus-responsive material is responsive to light, electric field, ultrasound, magnetic field, electromagnetic field, ionic strength, a change in pH, or a change in temperature,
wherein the second bioactive agent is the same as or different from the first bioactive agent, and
wherein, upon exposure to a stimulus, the stimulus-responsive material changes at least a property to cause the second bioactive agent to change its release profile.

35. The method of claim 34 wherein the first bioactive agent and the second bioactive agent are an antiproliferative, antiinflammatory or immune modulating, antimigratory, antineoplastic, antimitotic, antiplatelet, anticoagulant, antifibrin, antibiotic, antioxidant, antiallergic substances, or antithrombotic, or a pro-healing agent, or combinations of these.

36. The method of claim 34 wherein the first bioactive agent and the second bioactive agent are paclitaxel, docetaxel, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), tacrolimus, dexamethasone, rapamycin, rapamycin derivatives, 40-O-(2-hydroxy)ethyl-rapamycin (everolimus), 40-O-(3-hydroxy)propyl-rapamycin, 40-O-[2-(2-hydroxy)ethoxy]ethyl-rapamycin, and 40-O-tetrazole-rapamycin, 40-epi-(N1-tetrazolyl)-rapamycin (ABT-578), γ-hiridun, clobetasol, pimecrolimus, imatinib mesylate, or midostaurin, or prodrugs, co-drugs, or combinations of these.

37. A medical device comprising a coating formed of the method of claim 16.

38. A medical device comprising a coating formed of the method of.

39. A medical device comprising a coating formed of the method of claim 25.

40. A medical device comprising a coating formed of the method of claim 34.

41. The medical device of claim 1 which is a stent.

42. The medical device of claim 37 which is a stent.

43. The medical device of claim 38 which is a stent.

44. The medical device of claim 39 which is a stent.

45. The medical device of claim 40 which is a stent.

46. The medical device of claim 1 which is an absorbable stent.

47. The medical device of claim 37 which is an absorbable stent.

48. The medical device of claim 38 which is an absorbable stent.

49. The medical device of claim 39 which is an absorbable stent.

50. The medical device of claim 40 which is an absorbable stent.

51. A method of treating a disorder in a patient comprising implanting in the patient the medical device of claim 1, wherein the disorder is atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, tumor obstruction, or combinations thereof.

52. A method of treating a disorder in a patient comprising implanting in the patient the medical device of claim 37 wherein the disorder is one of atherosclerosis, thrombosis, restenosis, hemorrhage, vascular dissection or perforation, vascular aneurysm, vulnerable plaque, chronic total occlusion, claudication, anastomotic proliferation for vein and artificial grafts, bile duct obstruction, ureter obstruction, or tumor obstruction, or combinations of these.

53. The method of claim 51 further comprising exposing the stimulus-responsive material to an external stimulus.

54. The method of claim 53 further comprising exposing the stimulus-responsive material to an external stimulus.
